(19) United States
(12) Patent Application Publication
(10) Pub. No.: US 2017/0071536 A1
(43) Pub. Date: Mar. 16, 2017

(54) DYNAMIC BIOLOGICAL AND CHEMICAL SENSOR INTERFACES

(71) Applicant: BIOINK CORPORATION, San Francisco, CA (US)

(72) Inventors: Skylar Jackson Eagle Tibbits, Boston, MA (US); Carlos Edel Olguin, San Francisco, CA (US); Marcelo Henrique Coelho, Boston, MA (US); Tal Danino, New York, NY (US)

(21) Appl. No.: 15/358,415

(22) Filed: Nov. 22, 2016

Related U.S. Application Data
(63) Continuation-in-part of application No. 15/162,438, filed on May 23, 2016.
(60) Provisional application No. 62/258,498, filed on Nov. 22, 2015.
(60) Provisional application No. 62/165,493, filed on May 22, 2015.

(51) Int. Cl. A61B 5/00; G01N 31/22; G01N 33/52; B01L 3/00

(52) U.S. Cl. CPC A61B 5/4845; B01L 3/502715; G01N 31/22; G01N 33/525; A61B 5/6832; B01L 2300/0663; B01L 2300/0887; B01L 2300/041

(57) ABSTRACT

A dynamic sensor interface is provided. Such a dynamic sensor interface may include a reaction layer that includes a biological-based or chemical-based ink that reacts in response to a molecule of interest, a porous membrane that allows for the molecule of interest to pass through to a side that is in contact with the reaction layer, and an adhesive substrate. The reaction of the ink may include a change in a visual appearance of the ink, such as a change in color, transparency, movement within the reaction layer, three-dimensional expansion, three-dimensional contraction, or a tactile change. The reaction layer may include one or more microfluidic channels arranged in a predetermined arrangement such that movement through the microfluidic channels visually indicates detection of the molecule of interest. The reaction of the ink may further include a change in an olfactory property or a thermal property.

### CROSS-REFERENCE TO RELATED APPLICATIONS

The present application claims the priority benefit of U.S. provisional application No. 62/258,498 filed Nov. 22, 2015 and entitled “Dynamic Design Elements for Biological and Chemical User Interfaces,” the disclosure of which is incorporated herein by reference.

The present application is a continuation-in-part of U.S. patent application Ser. No. 15/162,438 filed May 23, 2016, which claims the priority benefit of U.S. provisional patent application 62/165,493 filed May 22, 2015, the disclosures of which are incorporated herein by reference.

### BACKGROUND OF THE INVENTION

Field of the Disclosure

This disclosure relates generally to sensor interfaces. In particular, the disclosure relates to biological-based and chemical-based sensor interfaces.

Description of the Related Art

Sensors may be used to detect various types of information regarding a person, object, or environment. Presently available sensors are generally mechanical in nature. Such mechanical sensors may include a variety of mechanical components based on the type of stimuli or other information being sensed, measured, and reported. As such, mechanical sensors are generally bulky, heavy, cumbersome, or otherwise uncomfortable and inconvenient to wear or carry. Smaller mechanical sensors, on the other hand, may lack accuracy or lack ability to convey much information beyond a binary indication. Such sensors may be unable, for example, to indicates changes that occur over time, changes in behavior, or other characteristics.

In addition, mechanical sensors may often require an external power sources (e.g., battery), which limits the scalability of the sensor given the bulky nature of batteries, wires, and other power input accessories. Similarly, power requirements constrain the applications given the risk of harming the user with overheating, failed batteries, shock or other types of electrical failure.

There is, therefore, a need in the art for improved systems and methods for dynamic sensor interfaces.

### SUMMARY OF THE CLAIMED INVENTION

A dynamic sensor interface is provided. Such a dynamic sensor interface may include a reaction layer that includes a biological-based or chemical-based ink that reacts in response to a molecule of interest, a porous membrane that allows for the molecule of interest to pass through to a side that is in contact with the reaction layer, and an adhesive substrate. The reaction of the ink may include a change in a visual appearance of the ink, such as a change in color, transparency, movement within the reaction layer, three-dimensional expansion, three-dimensional contraction, or a tactile change. The reaction layer may include one or more microfluidic channels arranged in a predetermined arrangement such that movement through the microfluidic channels visually indicates detection of the molecule of interest. The reaction of the ink may further include a change in an olfactory property or a thermal property.

### BRIEF DESCRIPTION OF THE DRAWINGS

FIG. 1 illustrates an exemplary structure for a wearable biological or chemical-based sensor interface.

FIG. 2A illustrates an exemplary reaction by a wearable biological or chemical-based sensor in response to detected stimuli or other information.

FIG. 2B illustrates another exemplary reaction by a wearable biological or chemical-based sensor in response to detected stimuli or other information.

FIG. 2C illustrates another exemplary reaction by a wearable biological or chemical-based sensor in response to detected stimuli or other information.

FIG. 2D illustrates another exemplary reaction by a wearable biological or chemical-based sensor in response to detected stimuli or other information.

FIG. 2E illustrates another exemplary reaction by a wearable biological or chemical-based sensor in response to detected stimuli or other information.

FIG. 3 is a diagram of an exemplary wearable biological or chemical-based sensor interface 100.

FIG. 4 illustrates an exemplary wearable sensor interface architecture and a variety of different sensor interface states.

### DETAILED DESCRIPTION

A dynamic sensor interface is provided. Such a dynamic sensor interface may include a reaction layer that includes a biological-based or chemical-based ink that reacts in response to a molecule of interest, a porous membrane that allows for the molecule of interest to pass through to a side that is in contact with the reaction layer, and an adhesive substrate. The reaction of the ink may include a change in a visual appearance of the ink, such as a change in color, transparency (including changing between visibility and invisibility), movement within the reaction layer, three-dimensional expansion, three-dimensional contraction, or a tactile change. The reaction layer may include one or more microfluidic channels arranged in a predetermined arrangement such that movement through the microfluidic channels visually indicates detection of the molecule of interest. The reaction of the ink may further include a change in an olfactory property or a thermal property.

Such a dynamic sensor interface may be temporarily adhered to a variety of different surfaces, including skin, clothing, packaging, etc. The reaction layer may include any combination of functional inks, which may be logically structured (e.g., programmed) to provide certain notifications or indicia when one or more stimuli of interest (e.g., molecules such as alcohol, medication, etc.) are detected with respect to internal or external environments. The functional inks—which may be biologically or chemically-based—react when a predetermined stimulus is detected, resulting in a variety of possible transformations. Such transformations may occur with respect to colors, shapes, textures, temperatures, smells, tastes, behaviors, etc., to display information to the user or others. The functional ink may be placed in specific arrangements to conceal or display information indicative not just of the stimulus of interest, but other information such as identity, environmental stimuli, biological or health information, exposure time, etc.

The sensor interface may therefore display information through many channels including optical means, such as visibility/invisibility or color change; physical means, such as movement or shape change from one state into another; a change in behavior (speed of movement, direction, degree of change etc.); or other means. Environmental stimuli may include temperature, light, moisture or other factors. User input may include applying pressure, licking, exhaling, uttering sounds, covering the ink, or administering more functional ink.

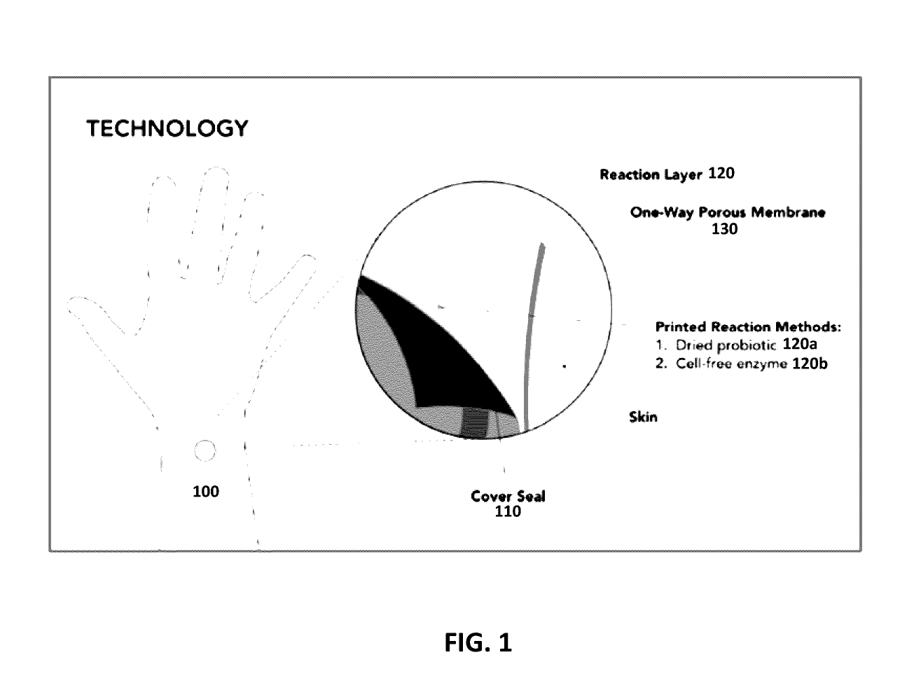

FIG. 1 illustrates an exemplary structure for a wearable biological or chemical-based sensor interface 100. The sensor interface 100 may be worn as a temporary tattoo decal placed on the skin. The sensor interface 100 may include several layers, including a cover seal 110, a reaction layer 120 (which may be made up of a dried probiotic material 120a or a cell-free enzyme material 120b), and a porous membrane layer 130.

The cover seal layer 110 is a layer that protects the other layers from damage and/or regulates their exchange with the external environment. The reaction layer 120 is the layer that may be printed with functional ink, which may be biological-based (e.g., bacterial, dried probiotic 120a) or chemical-based (e.g., cell-free enzyme 120b) or a combination of the same. Such functional ink may be formulated or otherwise engineered to sense a predetermined stimulus (e.g., molecule) of interest that may be found in sweat, other bodily triggers, or the external environment.

The porous membrane layer 130 may be in contact with a surface and may include an adhesive substrate that adheres to various surfaces (e.g., skin). The porous membrane layer 130 may serve as a porous membrane that allows sweat or other substances from the skin to pass through to the reaction layer 120, which may be prevented from contacting the skin directly by the porous membrane layer 130.

When the functional ink of reaction layer 120 detects the stimulus of interest, such ink may undergo a perceptible reaction. Such a reaction may include optical changes, movement or behavioral changes, tactile or shape changes, olfactory changes, and thermal changes.

Examples of optical changes may include changes in color, transparency (visibility), movement, behavior, shape, and three-dimensional contraction and expansion. A color change may occur, for example, when the functional ink of the reaction layer 120 comes into contact with a particular stimulus, resulting in biochemical reactions that change the color of the functional ink. Color transformation can happen across a range of hues, alpha levels (e.g., from opacity to transparency), or polarization (e.g., similar to an image on a computer screen).

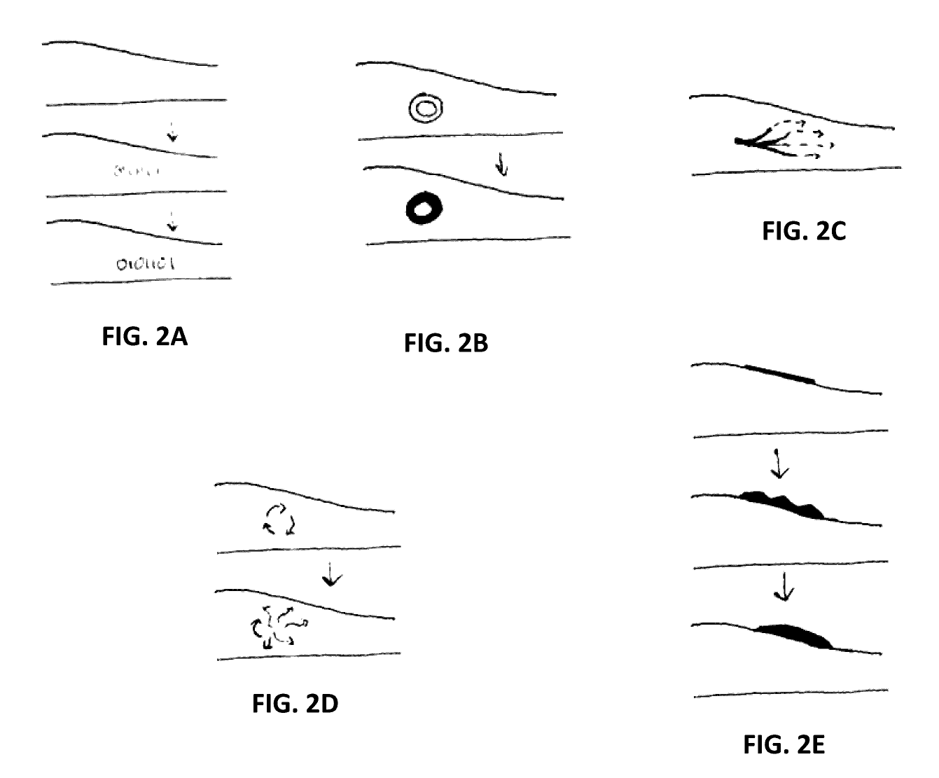

FIG. 2A illustrates an exemplary reaction by a wearable biological or chemical-based sensor interface 100 in response to detected stimuli or other information. As illustrated in FIG. 2A, the functional ink of the sensor interface 100 may initially appear clear or invisible against the surface to which it adheres before exposure to the stimulus of interest. As the stimulus is sensed, however, the functional ink in the sensor interface 100 may become visible. The functional ink may then become visible, for example, as sweat or alcohol from skin passes through porous membrane layer 130 to reaction layer 120. In some embodiments, the degree of opacity or transparency may be proportional to the amount of stimulus detected. In some instances, an ink may increase (e.g., fade in) or decrease (e.g., fade out) in prominence in response to a stimulus. As illustrated in FIG. 2A, the functional ink may be partitioned into a visual interface element.

FIG. 2B illustrates another exemplary reaction by a wearable biological or chemical-based sensor interface 100 in response to detected stimuli or other information. Similar to the sensor interface 100 reaction illustrated in FIG. 2A, the sensor interface 100 of FIG. 2B undergoes a reaction in which an initially invisible functional ink becomes visible. As further illustrated in FIG. 2B, the sensor interface 100 may include nonfunctional ink that appears as two concentric circles. As stimuli are sensed, however, the initially invisible functional ink partitioned between the two concentric circles becomes visible.

In some embodiments, visibility of the functional ink is relative to its background. For example, a particular functional ink may appear red before exposure to a certain stimulus (e.g., UV light), but become blue when exposed to UV light. Graphic interface elements—each utilizing different types of functional and nonfunctional inks partitioned within the reaction layer 120—can appear and disappear in relation to their background. A red flower (functional ink) on a red background (nonfunctional ink), for example, may initially appear invisible until exposed to UV light. The functional ink may then turn the flower blue against the background, which remains red.

In another embodiment, the functional ink may initially be visible before exposure to a stimulus, but disappear when the presence of the stimulus is detected. Depending on the selected functional ink(s) (and nonfunctional inks), such functional ink may either hide or visualize information as an indication or reminder regarding certain stimuli.

In another implementation, the sensor interface 100 may include one functional ink in channels arranged to form text (e.g. “NO”). As the sensor interface 100 detects a stimulus, the displayed result may be changed (e.g., “YES”). Such changes may be based on the structure of the channels, as well as the respective inks and ink attributes (e.g. movement, visibility/invisibility). For example, one ink may become invisible, while another ink—that was formerly invisible—may become visible within channels arranged into different text (e.g., “YES”). This allows the interface to transform in appearance into a completely different interface.

FIG. 2C illustrates another exemplary reaction by a wearable biological or chemical-based sensor interface 100 in response to detected stimuli or other information. When the functional ink senses stimuli, the ink may begin to move throughout embedded microfluidic channels in the reaction layer 120. Diffusing through such channels allow the functional ink to go from one configuration to another configuration, completely changing the appearance of the sensor interface. For example, a sensor interface may initially appear as a single line. When a stimulus (e.g., alcohol) is detected, the functional ink within the line may transform into a branching structure. In some cases, the number of branches may indicate a quantitative measurement of how much stimulus (e.g., alcohol) is detected.

When the functional ink senses stimuli in some embodiments, the ink may begin to move throughout embedded microfluidic channels in the reaction layer 120, demonstrating not only a change in appearance of the interface (e.g., moving through different channels) but a behavior change within the interface. The functional ink may begin to move faster, slower, more chaotically, change direction, oscillate, or demonstrate various other behavior changes.

FIG. 2D illustrates another exemplary reaction by a wearable biological or chemical-based sensor in response to detected stimuli or other information. A sensor interface 100 may initially appear, for example, in the form of a circle rotating clockwise when there is at least one microfluidic channel structured as a circle. As the sensor interface 100 detects a stimulus, however, the functional ink may escape the circular channel and move outward radially, appearing as a pulsing sunburst.

Changed behavior may occur when the ink is moving faster and more chaotically in reaction to the detected stimulus. In alternative embodiments, other functional inks may move more slowly in reaction to their respective stimuli. Other types of behavioral changes may include oscillation into smaller or larger circles and glowing (e.g., like a power indicator on an electronic device).

FIG. 2E illustrates another exemplary reaction by a wearable biological or chemical-based sensor interface 100 in response to detected stimuli or other information. When the functional ink senses stimuli, the ink may begin to change in a three-dimensional manner (e.g., bulging off the skin to create a structure or shape on the user's skin). Such three-dimensional changes may be produced as a result of embedded gasses within the functional ink or that may be produced by the functional ink as a result of stimulus detection. Such reactions may be used to create shapes for tactile notifications. Such functional ink may therefore for (Braille) notifications to visually-impaired users or for an additional level of transformation and embedded information.

Such shape transformation may be created through pressure changes (e.g., off gassing) within arranged channels within the reaction layer 120. Multiple functional inks with three-dimensional expansion or contraction capabilities may be arranged within the reaction layer to create a bimetal effect (e.g., curling or other complex three-dimensional shape changes).

A functional ink that responds to glucose concentration levels, for example, may have an initial state (e.g., prior to stimulus exposure) in which a relatively flat appearance is presented. As increasing glucose levels are detected, the functional ink may expand to create a physical texture indicative of the change in glucose level. The user may therefore be notified of such change via tactile sensation. An undulating 3-dimensional pattern may appear, for example. Further, when a secondary stimulus is sensed, the same or another function ink may create a bulge into a larger convex curve. Such three-dimensional properties may be likewise be arranged so as to present various types of notifications.

In some embodiments, the functional ink exhibits olfactory or thermal changes in reaction to a stimulus. When a functional ink contacts certain stimuli, a particular odor may be produced (e.g., off gassing) that is detectable by the wearer. The odor could be both pleasant, unpleasant or neutral depending on the kind of information the sensor interface seeks to communicate. When a functional ink detects a predetermined level of alcohol, for example, a banana smell may be emitted as a notification of the same.

Thermal changes may also occur when a function ink releases thermal energy to indicate to the user that a certain state has been reached. In some embodiments, the functional ink may detect that a certain level of alcohol has been reached and react to generate heat.

Using various combinations of functional inks—each reacting to specific stimuli in specific ways—a biochemical-based sensor interface may be logically structured (or “programmed”) to perform complex functions. The sensor interface may therefore represent a type of biochemical computing platform by which certain inputs (e.g., stimuli) may be detected and processed by functional ink(s), as well as result in useful biochemical transformations.

Functional inks may therefore be analogous to a network engineered as interconnected modules of Boolean circuits. These specialized intermediate compositions of inks may be selected and engineered to embody the behavior of a logical gate (e.g., NOT, AND, NAND, OR, etc.). Such a network may further be interconnected with others to create more sophisticated systems.

As noted above, the functional inks may be formulated or engineered to react with specific internal or external conditions to produce specific biochemical changes. For example, a functional ink may be engineered to detect alcohol (ethanol), glucose, electrolytes, and other conditions. Each functional ink may further have an initial, pre-exposure state, which differs from its respective post-exposure state. As noted above, such states may span various visual/optical, movement/behavioral, tactile/three-dimensional, olfactory, and thermal states. Such states can be set or unset, as well as transmitted or transduced internally or externally to create more sophisticated sensor-processor-actuator systems.

As such, functional inks can be selected based on their capability to oscillate between states. Some functional inks may change from state A to state B, as well as stay at state B indefinitely. For example, a functional ink may detect sunlight and become visible or otherwise visually transform from an initial state (e.g., plain circle) into another state (e.g., intricate sun pattern). Such functional ink may remain in the transformed pattern until the sensor interface 100 is removed.

Other functional inks may change from state A to B in the presence of stimuli and in the absence of stimuli, revert from state B to state A. For example, a functional ink may detect sweat-alcohol levels above the driving limit and appear as a red “STOP” pattern to indicate that the user is exhibiting elevated alcohol levels and should stop drinking. When the sweat-alcohol level drops below the legal limit, the red “STOP” notification may disappear.

Some functional inks may exhibit multiple discrete and continuous states. For example, a functional ink may transition through multiple states in a discrete fashion, from state A to state B, then from state B to state C, and then from state C back to state A. For example, a sensor interface for detecting ethanol may represent discrete levels of alcohol consumption as a set of stacked bars, including one or more bars designated as beyond a DUI threshold.

The transition may alternatively occur in a continuous fashion (e.g., along a gradient) to represent a plurality of conditions in relation to a stimulus. A sensor interface may include a combination of different functional inks, each detecting a different stimulus relevant to health. Such a sensor interface may therefore be able to reflect an overall state of health (e.g., normal, mild illness, critical) as it aggregates multiple stimuli (e.g., hydration, body temperature, cholesterol, glucose levels). Each stimulus may have a different weight based on arrangement of the respective functional inks in accordance with each stimulus' assigned impact on overall state of health.

In some embodiments, several functional inks may be combined in programming for a conditional compounded measurement. For example, a sensor interface 100 may include multiple functional inks. A first ink reacts in response to detecting a stimulus (e.g., alcohol), while a second ink reacts in response to detecting another (e.g., prescription medication that may have harmful side effects in the presence of alcohol). In the absence of medication, the first ink may simply produce an indication of alcohol levels. In the absence of alcohol, the second ink may produce an indication of the effectiveness (or other attribute) of the prescription medication. When both inks detect their respective substances, however, the sensor interface 100 may begin to display a warning symbol.

Another type of combination may allow for path-dependent measurement (e.g., hysteresis). For example, a sensor interface 100 may also show different states in a conditional fashion—whether compounded or not—when exposed to the same stimuli based on the value of the previous state(s). When so programmed, a glucose biosensor interface may result in a first pattern of a red arrow facing up when glucose levels are high and increasing; when glucose levels are high and decreasing, however, a red arrow facing down may be displayed.

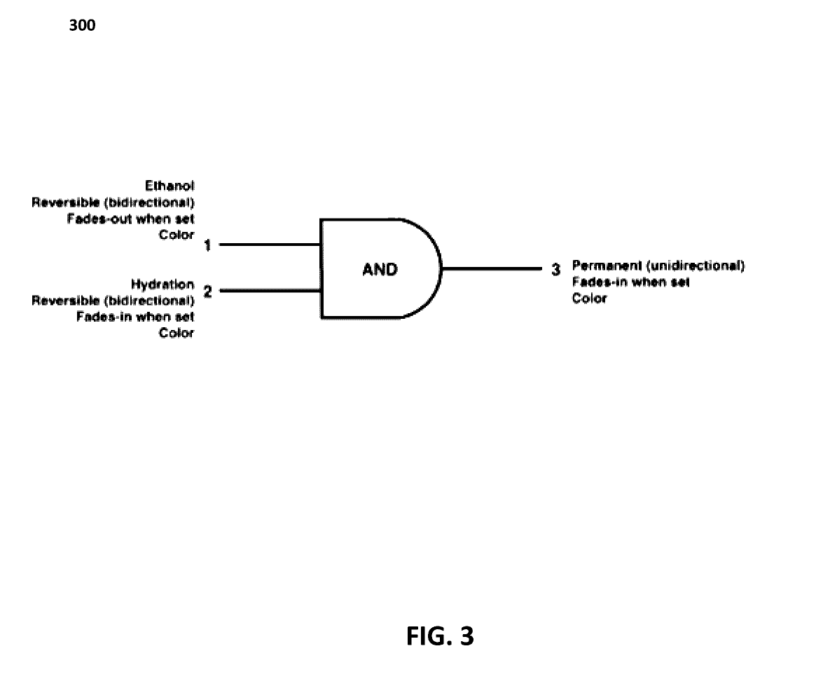

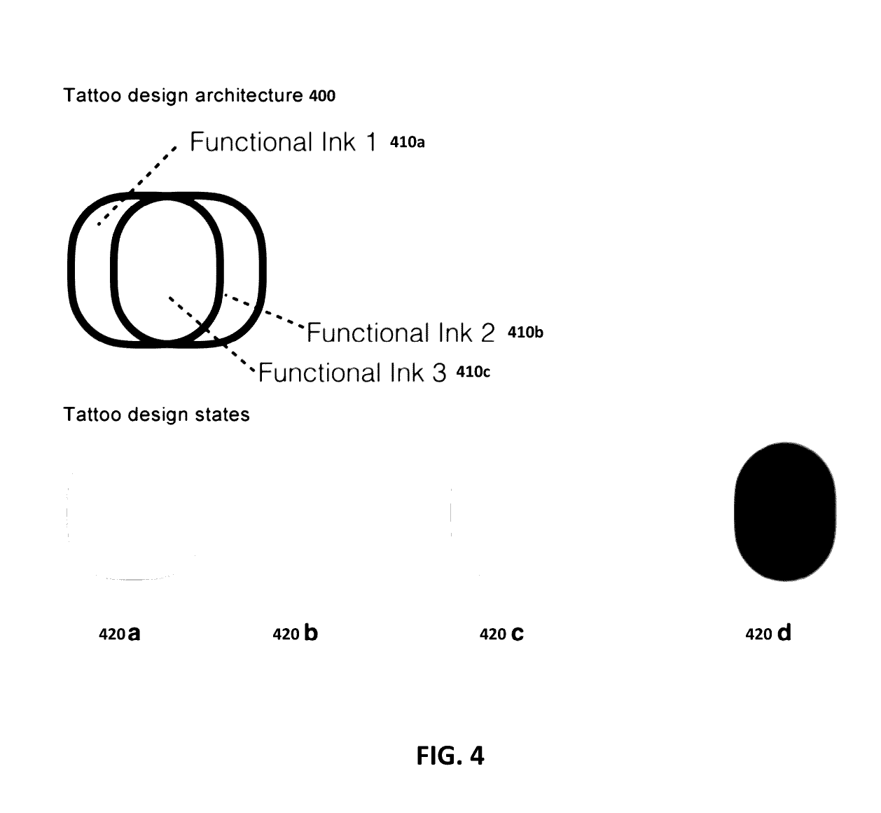

FIG. 3 is a diagram 300 of an exemplary wearable sensor interface 100, and FIG. 4 illustrates the exemplary wearable sensor interface architecture 400 and a variety of different sensor interface states 420a-d. Such a sensor interface 100 may include three inks: (1) a first ink 410a for detecting alcohol (ethanol) and with an unset state (pre-exposure) where a color is exhibited prominently and a set state (exposure at levels above a predetermined DUI impairment level) where the color fades in prominence; (2) a second ink 410b for detecting hydration level and with an unset state where color fades in prominence when hydration levels are normal and a set state where color increases in prominence when hydration levels fall below a predefined threshold of normalcy; (3) a third ink 410c for detecting the reactions of both of the first two inks (410a and 410b) and with an unset state where no or little color is exhibited and a set state where color increases in prominence.

State 420a is one in which all inks are unset and only the first ink 410a is visible. In state 420a, no alcohol has been detected, and hydration levels are normal. In state 420b, only the first ink 410a is set, indicating that alcohol has been detected but hydration levels are still normal. In state 420c, only the second ink 410b is set, indicating that hydration levels are below the predefined normal level but no alcohol has been detected. Finally, in state 420d, all three inks 410a-c are set, indicating that both alcohol and below-normal hydration levels are detected.

As such, the third ink reacts when both alcohol and low hydration levels are detected. Whereas the first ink and the second ink may be reversible (e.g., bi-directional), the third ink may be permanently set (e.g., unidirectional). As such, the third ink may only be reset by replacing the sensor interface 100. The first ink may be programmed to serve as a less conspicuous notification of DUI levels, since the notification is the absence of color.

The way in which a functional ink transitions from one state to another may be analogous to an LED control. Such transition may be characterized along a sinusoidal wave, square wave, saw tooth, etc. The speed of transition can be immediate or gradual depending on both the levels of detection, transformation speed, external temperature, or other selected factors. For example, a color-changing function ink can oscillate between two different colors at 0.5 Hz to indicate a high level of alcohol concentration, as well as oscillate at 0.1 Hz to indicate a low level of alcohol concentration. In addition, such oscillation can transition from a sinusoidal transformation to a saw tooth one, where the sharper edges in color change may become more visible to the user and serve as an indication that she or he should stop drinking.

A current state may be compared to a previous state by structuring different channels with different reactions. Alternatively, a comparison may be obtained by providing an initial condition spot (e.g., the control) and a secondary spot for the current reaction. The color, shape, movement, or behavior of the ink at the secondary spot can be compare to the control so as to evaluate the extent to which the current condition differs. The user may want to know exactly how different the current state has changed from the initial state over time. For example, two small ink circles may be provided on a sensor interface 100. One circle may remain constant, while the other circle changes in the presence of a stimulus, allowing for the comparison between the two.

Functional ink can be used to show a change over time, similar in ways to a time-lapse or clock without the need for a comparison to a control. The movement, color change, transparency, or behavior change can be used to indicate the speed or gradual transformation. For example, an ink circle may be provided with incremental channels structured like a clock around the circumference of the circle. As the amount of input stimulus increases, the ink gradually changes color in a clockwise pattern around the circle, filling each of the incremental channels. As such, the ink indicates an amount of stimulus that is sensed over time.

Further, ink can be used to show the speed of a reaction or speed of change with the increase of a stimulus. For example, a circular channel of ink may detect a stimulus and begin to move in a clockwise manner around the channel. When the amount of stimulus increases, the ink may move faster around the circle at a rate corresponding to the speed of change and increase in the quantity of the stimulus.

Functional ink can also be used to show accumulated results, not only the binary state or the speed of a reaction. The user can look at the sensor interface to understand how much of something has been sensed. For example, an ink grid with individual chambers may sense a stimulus, and each one of the individual chambers may change color in an incremental manner based on the quantity or reaction levels sensed. The grid may change color from left-to-right or top-to-bottom, showing an aggregated amount sensed rather than just a snapshot.

A series of channels may be created in the reaction layer 120 and filled with functional ink. Such a series of channels may serve as logic gates so that when a stimulus is sensed, the logic gates become activated. As the functional ink moves through the gates, the resulting reaction adds an amount of input and provides a result in the form of a binary number.

Functional ink can be used to show or conceal private information. A functional ink with a transparency, color, movement, behavior, or other change can help to display information only when subject to a specific stimulus. In this way, the sensor interface can act as a barcode or a hidden user ID. For example, a sensor interface design may indicate a user's blood type, social security number, medical information, or other private information only when a very specific stimulus is applied (e.g., by a medical provider). Similarly, this could be used for validation, authentication, anti-counterfeiting protection, or sending private information from one party to another.

Functional inks can be used in combination with one another or in specific patterns (e.g., to test multiple stimuli at the same time). Rather than providing multiple sensors, a single combination sensor with different inks can be used to test for the presence multiple difference stimuli, thereby facilitating and speeding up testing. Similarly, such a combination sensor can indicate the simultaneous presence of multiple stimuli. Such a combination sensor may be implemented in the medical field, for example, in cases where a patient is being tested for different substances, whether natural or foreign.

Functional ink tattoos can be used to produce another substance in the presence of a stimulus. The reaction created when the sensor interface senses a stimulus can create energy, off gassing, other inks, or some other form of production. For example, a functional ink may sense a stimulus and react by off-gassing, thereby inflating the sensor interface into a 3-dimensional shape. The gas may include a perfume or cologne that is released over time so as to prolong their fragrance over time.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A dynamic sensor interface comprising:
a reaction layer comprising a biological-based or chemical-based ink that reacts in response to a molecule of interest;
a porous membrane that allows for the molecule of interest to pass through to a side that is in contact with the reaction layer; and
an adhesive substrate.

2. The dynamic sensor interface of claim 1, wherein the reaction of the ink includes a change in a visual appearance of the ink.

3. The dynamic sensor interface of claim 2, wherein the change in appearance includes at least one of a change in color, transparency, movement within the reaction layer, three-dimensional expansion, three-dimensional contraction, and a tactile change.

4. The dynamic sensor interface of claim 3, wherein the reaction layer includes one or more microfluidic channels arranged in a predetermined arrangement, and wherein the movement within the reaction layer includes movement through the one or more microfluidic channels to visually indicate detection of the molecule of interest.

5. The dynamic sensor interface of claim 3, wherein the movement within the reaction layer occurs at a rate or pattern that visually indicates detection of the molecule of interest.

6. The dynamic sensor interface of claim 3, wherein the reaction layer includes one or more gasses embedded in the ink, and wherein the reaction of the ink includes a pressure change to the one or more gasses to result in at least one of the three-dimensional expansion, three-dimensional contraction, tactile change, change in olfactory property, and change in thermal property.

7. The dynamic sensor interface of claim 6, wherein the pressure change occurs by offgassing of the one or more embedded gasses.

8. The dynamic sensor interface of claim 1, wherein the reaction of the ink is proportional to a detected amount of the molecule of interest.

9. The dynamic sensor interface of claim 8, wherein the reaction occurs along a gradient corresponding to the detected amount of the molecule of interest.

10. The dynamic sensor interface of claim 8, wherein the reaction includes an ordered sequence of states, each state corresponding to a different amount of the molecule of interest.

11. The dynamic sensor interface of claim 1, wherein the ink reverts to a former state when the molecule of interest is no longer detected.

12. The dynamic sensor interface of claim 1, wherein the reaction of the ink includes a change in an olfactory or thermal property of the ink.

13. The dynamic sensor interface of claim 1, wherein the reaction layer further includes one or more other inks that each detects a different stimulus of interest.

14. The dynamic sensor interface of claim 13, wherein each respective reaction of each ink to the respective stimulus of interest is weighted to produce a compound notification regarding the one or more stimuli of interest being detected.

15. The dynamic sensor interface of claim 13, wherein a combination of reactions by at least two ink results in a different reaction than each ink individually.

16. The dynamic sensor interface of claim 1, wherein the reaction of the ink is further indicative of a former state of the ink.

17. The dynamic sensor interface of claim 1, further comprising a cover seal layer.
